(19) United States
(12) Patent Application Publication
(10) Pub. No.: US 2015/0038588 A1
(43) Pub. Date: Feb. 5, 2015

(54) COMPOSITIONS AND METHODS OF TREATMENT OF STATUS EPILEPTICUS

(71) Applicant: YISSUM RESEARCH DEVELOPMENT COMPANY OF THE HEBREW UNIVERSITY OF JERUSALEM LTD., Jerusalem (IL)

(72) Inventors: Meir Bialer, Jerusalem (IL); Boris Yagen, Jerusalem (IL)

(21) Appl. No.: 14/383,228

(22) PCT Filed: Mar. 6, 2013
(86) PCT No.: PCT/IL2013/050204

§ 371 Date: Sep. 5, 2014

Related U.S. Application Data
(60) Provisional application No. 61/718,421, filed on Oct. 25, 2012.
(60) Provisional application No. 61/607,126, filed on Mar. 6, 2012.

(51) Int. Cl. A61K 31/16

(52) U.S. Cl. CPC A61K 31/16
USPC 514/629

(57) ABSTRACT

The invention provides a method of treating benzodiazepine-resistant status epilepticus in a subject having been exposed to a nerve agent inducing the status epilepticus.

### FIELD OF THE INVENTION

The invention is directed to a method of treatment of status epilepticus by treatment after onset of the condition.

### BACKGROUND

Status epilepticus, SE, is a life-threatening neurologic disorder requiring immediate treatment. There are two forms of SE: a generalized convulsive status epilepticus (GCSE), involving prolonged seizures; and non-convulsive status epilepticus (NCSE), involving changes in behavior, memory, affect or level of consciousness.

Status epilepticus has classically been defined as a prolonged seizure without recovery of consciousness or repetitive seizures lasting longer than 30 minutes. However, it is now accepted in the field that seizures persisting for more than several minutes are to be considered “impending” SE and therefore are generally treated aggressively.

Numerous etiologies underline SE, but in the last decade particular attention has been directed at the problem of how to treat patients with SE during a mass nerve-agent exposure, particularly since one of the most serious complications of nerve agents and other organophosphate poisons is SE. Furthermore, a mass nerve-agent exposure could affect thousands of individuals simultaneously, particularly children, and thus could be a large-scale medical emergency with profoundly adverse conditions for first-responders.

Two inter-related concerns in relation to a nerve-agent exposure are that SE becomes progressively refractory to medical treatment over time, and that it is likely that over 30 minutes (and potentially over 60 minutes) it will require first responders to begin to effectively treat nerve-agent victims. Treatment of SE typically begins immediately after diagnosis but the longer SE is allowed to progress without a treatment, the greater the risk for neurologic morbidity and a reduced responsiveness to medication. Thus, neurological outcomes generally depend on the severity and duration of SE.

Pharmacotherapy for SE generally involves intravenous administration of three classes of drugs:

(1) benzodiazepines for rapid control of SE via GABA_{A} receptors,

(2) traditional antiepileptic drugs (AEDS) aimed at additional molecular mechanisms and more long-term coverage, and

(3) general anesthetics.

Benzodiazepines such as diazepam (DZP) are generally considered first-line therapy, but traditional antiepileptic drugs (AEDs) including phenytoin and valproic acid (VPA) are second-line therapy for refractory SE [1]. The anesthetics propofol and pentobarbital provide a third-line of therapy. First- and second-line therapies often do not suppress electrographic SE (ESE), and third-line therapies cannot be administered in the field.

The use of nerve agents for experimentation is highly restricted for security reasons and limited to specific research sites. Nerve agents cause diverse systemic effects that can confound quantitative analyses of drug actions on repetitive seizures and ESE. A widely used and particularly severe animal model of ESE that is useful to model nerve-agent exposure [2] involves single-dose intraperitoneal treatment with pilocarpine, preceded by lithium. Electrographic activity after lithium-pilocarpine treatment has been used to model the severe ESE that can result from nerve-agent exposure.

Because it is well-established that non-convulsive ESE can persist after aggressive pharmacological treatment, prolonged and continuous EEG recording has become increasing important in preclinical research on animal models [3], in clinical studies on the efficacy of anti-seizure therapeutic agents, and in the diagnosis of ESE [4].

Valproic acid (VPA) is the least potent AED, and its clinical use is limited by hepatotoxicity and teratogenicity; thus, numerous VPA analogues and derivatives have been designed and evaluated [5]. One of these is sec-butyl-propylacetamide (SPD), which is a homologue of valnoctamide (VCD), a chiral constitutional isomer of VPA's corresponding amide valpromide (VPD) [6,7]. Pouliot et al., [8] reported on a comparative electrographic analysis of the effect of sec-butyl-propylacetamide on pharmacoresistant status epilepticus.

VCD having the formula below:

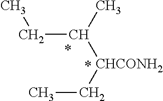

is a CNS-active chiral constitutional isomer of valpromide, the corresponding amide of valproic acid (VPA) that exhibits stereoselective pharmacokinetics (PK) in humans and animals [5].

VCD, being an amide analogue of VPA was found to have anti-convulsant activity and was further found to be distinctly less teratogenic than VPA [9].

White et al., [7] tested the effects of various anti-epileptic drugs on lithium pilocarpine status epilepticus and showed that while VCD was equivalent to SPD when given at SE onset, it lost its activity at 80 mg/kg when administered 30 minutes after SE onset. The authors also showed that the anticonvulsant carbamazepine was shown to block pilocarpine induced convulsive SE seizures (ED50=50 mg/kg) at 30 minutes post SE seizures.

### REFERENCES

[1] Abend and Dlugos., Pediatr Neurol. 2008; 38: 377-390.
[2] Tang F R, et al., Curr Med Chem. 2011; 18:886-899.
[3] Lehmkuhle M J, et al., J Neurophysiol. 2009; 101: 1660-1670.
[4] Bautista R E, Godwin., SCaro, D. J Clin Neurophysiol. 2007; 24: 16-21.
[5] Bialer and Yagen., Neurotherapeutics. 2007; 4: 130-137.
[6] Kaufmann D, et al., Neuropharmacology. 2010; 58: 1228-1236.
[7] White, H S, et el., Epilepsia. 2012. 53:134-146.
[8] POULIOT, M, et al., Neuroscience. 2013; 231: 145-156.
[9] Radatz M, et al., Epilepsy Res. 1998; 30(1):41-8.

### SUMMARY OF THE INVENTION

As mentioned hereinabove, it was previously found that VCD is equipotent to SPD when given at SE onset, but in contrast to SPD, VCD lost its activity when administered 30 minutes after the SE onset. Thus, there exists a need in the field for an effective therapy to treat SE after its onset, namely at a point in time after a subject exposed to the nerve gas has begun to exhibit symptoms associates with SE (e.g. seizure motor activity, hallucinations, coma, lethargy, confusion), and more so at points of time after nerve gas exposure where traditional treatments (e.g. benzodiazepines alone or combined with antiepileptic drug) has failed.

The present invention is based on the surprising finding that contrary to what was expected based on the results obtained by using VCD for treating SE after its onset, when given at high enough doses, VCD was effective in the treatment of SE when administered long after seizure onset. Thus, VCD was found suitable to effectively treat nerve-agent victims minutes and hours after exposure.

Thus, in one aspect of the invention, there is provided a method of treating benzodiazepine-resistant status epilepticus (SE) in a subject having been exposed to a nerve agent inducing said SE, the method comprising administering to the subject a therapeutically effective amount of valnoctamide (VCD) or a pharmaceutically acceptable salt thereof, wherein said VCD is administered after said subject has experienced at least one SE episode indicative of exposure to said agent.

In the context of the present invention, the SE may be treated by administering to the subject an effective amount of valnoctamide, VCD, 2-ethyl-3-methyl-pentanamide, having the formula (I):

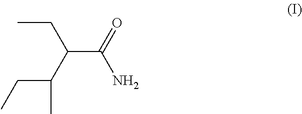

The term “VCD” also refers to any of VCD's 4 stereoisomers, in pure form, ((2S,3S)-VCD, (2S,3R)-VCD, (2R,3R)-VCD and (2R,3R)-VCD; to combinations of two or three of the enantiomers in any relative ratios; to combination of four enantiomers in ratios other than 25% each (non-racemic mixtures) as well as to a racemic mixture (a mixture containing equal amounts of each of the 4 stereoisomers of VCD).

“Status epilepticus (SE)” refers to a life-threatening condition in which the brain is in a state of uncontrolled persistent seizures. More specifically, SE is defined as a continuous seizure lasting at least 5 minutes (and in some cases more than 2 minutes) and typically more than 30 minutes or two or more seizures without full recovery of consciousness between any of them. Prolonged SE can lead to cardiac dysrhythmia, metabolic derangements, autonomic dysfunction, neurogenic pulmonary edema, hyperthermia, rhabdomyolysis, and pulmonary aspiration. Permanent neurologic damage can occur with prolonged SE.

As used herein, the “nerve agent inducing SE” is generally a nerve agent selected from a class of phosphorus-containing organic chemicals that disrupt the mechanism by which nerves transfer messages to organs. Some none limiting examples of nerve agents, in accordance with the present invention, are: tabun (GA), sarin (GB), soman (GD), cyclosarin (GF), 2-(dimethylamino)ethyl N,N-dimethylphosphoramidofluoridate (GV), a Novichok agent, S-(diethylamino)ethyl O-ethyl ethylphosphonothioate (VE), O,O-diethyl S-[2-(diethylamino)ethyl]phosphorothioate (VG), 2-(ethoxymethyl phosphoryl) sulfanyl-N,N-diethyl ethanamine (VM), N,N-diethyl-2-(methyl-(2-methylpropoxy)phosphoryl)sulfanyl ethanamine (VR) and ethyl({2-[bis(propan-2-yl)amino]ethyl}sulfanyl)(methyl)phosphinate (VX), as well as various insecticides such as the phenothiazines, organophosphates such as dichlorvos, malathion and parathion (or its active metabolite paraoxon).

In accordance with the present invention, the “SE episode indicative of exposure” to said agent refers to a seizure (focal or generalized) or any physical reaction associated therewith (or with SE), which results from exposure to a nerve agent. Some none limiting examples of such SE episodes include: nystagmus or brief twitching of the face, eyelids, jaw, trunk, arms, hands, legs or feet (in the form of unilateral, intermittent and/or simulating focal seizures); muscle contraction or the tonic phase, followed by a phase of alternate contraction and relaxation of muscles or the clonic phase; a generalized seizure (e.g. characterized by bilateral synchronous limb movements); a focal seizure (e.g. characterized by movement of one or more extremities becoming secondarily generalized; autonomic disturbances (e.g. in the form of a tachycardia, cardiac arrhythmia, hypertension, high fever, salivation, vomiting and incontinence); unresponsiveness; ocular motor abnormalities; prolonged postictal confusion and unexplained coma.

In some embodiments, the SE episode indicative of exposure to said agent is a focal or generalized seizure.

In some embodiments, SE is not pilocarpine or lithium-pilocarpine induced SE.

In some embodiments, status epilepticus is induced by pilocarpine (PC) or lithium-PC in murine. As readily recognized by the skilled artesian, PC is a muscarinic receptor agonist that is used for the induction of experimental models of status epilepticus (SE) for studying the type of seizure-induced brain injury and other neuropathophysiological mechanisms of related disorder. Thus, in accordance with such embodiments, SE induced by PC and/or lithium-PC serves as a model to emulate exposure to nerve agents in humans such that the vascular and neurodegenerative phenomena (e.g. necrotic processes, apoptotic cell death) observed in the murine following intraperitoneal (I.P) injection of PC, as described herein, are predictive of the corresponding physiological response to be observed in humans following exposure to nerve agents.

As stated hereinabove, the SE treatable in accordance with the invention is a “benzodiazepine-resistant status epilepticus (SE)” (interchangeable with “refractory SE (RSE)”), defined as status epilepticus that continues despite treatment with at least one benzodiazepine (e.g. lorazepam, midazolam, diazepam) alone or in combination with at least one antiepileptic drug. In some embodiments, benzodiazepine-resistant SE is SE that has continued for longer than 20 minutes despite treatment with at least one benzodiazepine (e.g. lorazepam, midazolam, diazepam) alone or in combination with at least one antiepileptic drug.

Seizures typical to benzodiazepine-resistant SE may lead to coma and even to death in the absence of immediate treatment with an intravenous seizure suppressive agent. In the context of the present invention, benzodiazepine resistant SE also encompasses SE seizures that are pharmacologically refractory to treatment with pentobarbital, midazolam, thiopental, propofol or ketamine.

Treatment with VCD in accordance with the invention is provided to the subject after said subject has experienced at least one SE episode indicative of exposure to said agent, namely at any time after the occurrence of the first seizure, i.e. a seizure characterized by one continuous, unremitting seizure lasting longer than 5 minutes and/or after at least one SE episode (e.g. focal or generalized seizure) indicative of exposure to the nerve agent, as defined herein; thus, the treatment may be administered at least 30 minutes after said subject has experienced at least one SE episode indicative of exposure to said agent. In some embodiments, treatment is administered between 30 and 60 minutes after said subject has experienced at least one SE episode indicative of exposure to said agent, or between 45 and 60 minutes after said subject has experienced at least one SE episode indicative of exposure to said agent, or after 60 and 90 minutes after said subject has experienced at least one SE episode indicative of exposure to said agent, or between 1 hour and 2 hours after said subject has experienced at least one SE episode indicative of exposure to said agent, or after 2 hours after said subject has experienced at least one SE episode indicative of exposure to said agent.

In some embodiments, treatment is administered 30 minutes after said subject has experienced at least one SE episode indicative of exposure to said agent or at any time thereafter.

The time of treatment is not at zero time, namely is not immediately after exposure to a nerve agent.

The term “treatment” or any lingual variation thereof is used herein to indicate treating any SE associated condition (e.g. seizure, convulsion), or ameliorating, alleviating, reducing, or suppressing SE or slowing or stopping the progress of SE and/or any condition associated therewith, at any time after onset of SE is defined.

The invention further provides a method of treating benzodiazepine-resistant SE in a subject in need thereof, the method comprising administering to the subject a therapeutically effective amount of VCD or a pharmaceutically acceptable salt thereof, wherein the therapeutically effective amount of VCD is more than the human equivalent of 80 mg/kg in rats. In some embodiments, treatment by VCD is administered after onset of seizures, as defined hereinabove.

The “human equivalent” indicated, interchangeable with a human equivalent dose (HED), refers to the dose that produces in human the same effect as featured after administration of a specific dose of VCD in rats. The “effective amount” of VCD that should be administered to humans, for purposes herein, may be determined by determining the human equivalent, as detailed herein or by other considerations as may be known in the art. The amount must be effective to achieve the desired therapeutic effect as described above, i.e., treat SE after onset, depending, inter alia, on the type and severity of the symptoms and the treatment regime. The effective amount is typically determined in appropriately designed clinical trials (dose range studies) and the person versed in the art will know how to properly conduct such trials in order to determine the effective amount. As readily recognized by a person of skill in the art, the therapeutically effective amount of VCD depends and may be determined on the basis of a number of parameters such as body mass, age, gender, body surface area, absorption rate of VCD, clearance rate of VCD, rate of metabolism and any other parameters that may affect either the absorption or the elimination of VCD.

In some embodiments, the therapeutically effective amount of VCD is between the human equivalent of about 100 mg/kg and about 500 mg/kg in rats. In other embodiments, the therapeutically effective amount of VCD is between the human equivalent of about 100 mg/kg and about 200 mg/kg in rats. In still other embodiments, the therapeutically effective amount of VCD is the human equivalent of about 180 mg/kg in rats.

It is well known in the pertinent field of the art that an amount of 180 mg/kg administered to rats can be converted to an equivalent amount in another species (e.g. humans) by the use of one of possible conversion equations well known in the art. Examples of conversion equations from rats to humans are division of the rat doses by 6.2 to convert to human dosage.

For example, a human equivalent of more than 80 mg/kg is more than 13 mg/kg in humans, the equivalent of 100-500 rat mg/kg is 16-80 mg/kg in humans, the equivalent of 100-200 mg/kg in rats is 16-32 mg/kg in humans, and the human equivalent of 180 mg/kg in rats is about 30 mg/kg in humans.

As mentioned herein, the human equivalent may be calculated based on a number of conversion criteria as explained below or may be a dose such that either the plasma level will be similar to that in the murine (e.g. rat) following administration at a dose as specified hereinabove; or a dose that yields a total exposure (namely area under the plasma drug concentration versus time curve or AUC) that is similar to that in murine at the specified dose range.

In accordance with the present invention, the human equivalent to the murine dose may also be extrapolated to a human equivalent dose by using various parameters as readily recognized by the skilled artesian, e.g. body surface area (BSA) normalization method oxygen utilization, caloric expenditure, basal metabolism, blood volume, circulating plasma proteins renal function (as described, for example, in Reagan-Shaw et al., The FASEB Journal. vol. 22 no. 3 659-661).

In another aspect of the present invention, there is provided a pharmaceutical composition, comprising as an active agent a therapeutically effective amount of valnoctamide (VCD) or a pharmaceutically acceptable salt thereof for use in treating benzodiazepine-resistant SE in a subject having been exposed to a nerve agent inducing said SE, wherein said VCD is administered after said subject has experienced at least one SE episode indicative of exposure to said agent.

The composition of the invention may additionally comprise at least one inert agent selected from a buffering agent, an agent which adjusts the osmolarity thereof, a pharmaceutically acceptable carrier, excipient and/or diluents.

The pharmaceutically acceptable carriers, vehicles, adjuvants, excipients, or diluents, are well-known to those skilled in the art and are readily available to the public. It is preferred that the pharmaceutically acceptable carrier be one which is chemically inert to VCD and one which has no detrimental side effects or toxicity under the conditions of use.

The choice of a carrier will be determined in part by the particular active agent, as well as by the particular method used to administer the composition. The carrier can be a solvent or a dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants.

Suitable preservatives and buffers can be used in the compositions of the invention. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The requirements for effective pharmaceutical carriers for injectable compositions are well known to those of ordinary skill in the art. See Pharmaceutics and Pharmacy Practice, J.B. Lippincott Co., Philadelphia, Pa., Banker and Chalmers, eds., pages 238-250 (1982), and ASHP Handbook on Injectable Drugs, Toissel, 4^{th} ed., pages 622-630 (1986).

In still another aspect of the present invention, there is provided use of valnoctamide (VCD) or a pharmaceutically acceptable salt thereof in the preparation of a pharmaceutical composition for treating benzodiazepine-resistant SE in a subject having been exposed to a nerve agent inducing said SE, wherein said VCD is administered after said subject has experienced at least one SE episode indicative of exposure to said agent.

In still yet another aspect of the present invention, there is provided a pharmaceutical composition, suitable for injection, comprising a vehicle in which a therapeutically effective amount of VCD or at least one analog thereof is solubilized, said vehicle comprising between about 0.1 and 0.6 gram/1,000 ml of calcium chloride, between about 0.1 and 0.6 gram/1,000 ml of potassium chloride and between about 2 and 12 gram/1,000 ml of sodium chloride.

The “vehicle” is a solution which is suitable to solubilize the herein defined VCD or at least one analog thereof. In accordance with the present, the vehicle may include one or more suspending agents, one or more bulking agents, one or more buffers, and optionally one or more pH adjusting agents. Some none-limiting examples of suspending agents suitable for use in accordance with the present invention are sodium carboxymethyl cellulose, hydroxypropyl cellulose, carboxymethyl cellulose, hydroxypropylethyl cellulose, hydroxypropylmethyl cellulose, and polyvinylpyrrolidone in combination with sodium carboxymethyl cellulose.

In some embodiments, the suspending agent is polyvinylpyrrolidone.

In accordance with the present invention, the suspending agents may also comprise various polymers, low molecular weight oligomers, natural products, and surfactants, including nonionic and ionic surfactants. Most of these suspending agents are known pharmaceutical excipients and are described in detail in the Handbook of Pharmaceutical Excipients, 7^{th} edition, incorporated herein by reference. The suspending agents are commercially available and/or can be prepared by techniques known in the art.

Some none-limiting examples of buffers that are suitable for use in accordance with the present invention include sodium phosphate, potassium phosphate, and tris(hydroxymethyl)aminomethane (TRIS) buffer.

Examples of bulking agents that are suitable for use in accordance with the present invention include, but are not limited to, mannitol, sucrose, maltose, xylitol, glucose, starches, 15 sorbital, and the like.

Examples of pH adjusting agents include, but are not limited to hydrochloric acid or acetic acid. When the pH needs to be raised, a basic pH adjusting agent may be employed such as sodium hydroxide, potassium hydroxide, calcium carbonate, magnesium oxide or magnesium hydroxide.

In some embodiments, said vehicle comprising between about 0.2 and 0.4 gram/1,000 ml of calcium chloride, between about 0.2 and 0.4 gram/1,000 ml of potassium chloride and between about 6 and 10 gram/1,000 ml of sodium chloride.

In other embodiments, said vehicle comprising about 0.32 gram/1000 ml of calcium chloride, about 0.3 gram/1,000 ml of potassium chloride and about 8.6 gram/1,000 ml of sodium chloride.

As used herein, the VCD analog refers to any VCD analogs known in the art such as, but not limited to, valproic acid or an amide, N-methylamide and urea derivative thereof (as described for example in Kaufmann et al., J. Med. Chem. 2009, 26; 52(22):7236-48).

In some embodiments, said VCD analog is selected from valpromide VPD, ropylisopropyl, propylisopropyl acetamide (PID), and diisopropyl acetamide (DID).

The term “suitable for injection” is used to indicate that the herein described composition may be injected via the intramuscular (I.M), intraperitoneal (I.P), intradermal or subcutaneous (S.C) routes. In some embodiments, the composition of the invention is suitable for injection via the intramuscular route. When injected intramuscularly, the volume of a single injection is in the range of between about 0.1 ml and between about 3.5 ml.

As readily recognized by the skilled artesian, I.M injection is typically administered to the deltoid muscle of the arm, the vastus lateralis muscle of the leg, and the ventrogluteal and dorsogluteal muscles of the buttocks or to other body regions that are suitable for I.M injection.

In some embodiments, the composition is administered 30 minutes or more after said subject has experienced at least one SE episode indicative of exposure to said agent.

In some embodiments, the composition comprises a therapeutically effective amount of VCD or at least one analog thereof being more than the human equivalent of 80 mg/kg in rats. In some embodiments, the composition comprises a therapeutically effective amount of VCD or at least one analog thereof being between the human equivalent of about 100 mg/kg and about 500 mg/kg in rats.

In some embodiments, the composition comprises a therapeutically effective amount of VCD or at least one analog thereof being between the human equivalent of about 100 mg/kg and about 200 mg/kg in rats.

In some embodiments, the composition comprises a therapeutically effective amount of VCD or at least one analog thereof being the human equivalent of about 180 mg/kg in rats.

In some embodiments, the VCD or at least one analog thereof in the composition is in lyophilized form for the reconstitution in a sterile solution.

In some embodiments, the concentration of VCD or at least one analog thereof in the vehicle is between about 0.5 and 25% by weight in solution.

By yet another aspect, the present invention provides a kit (or a commercial package) for administration of a composition of the invention, said kit comprising:

a) an amount of VCD or at least one analog thereof, as defined herein;

b) a vehicle or solution for solubilizing said VCD; and

c) instructions of use.

The components composed in a kit according to the invention, may be contained in a single vessel or holding unit or in separate vessels and contain a label attached to or packaged with the container that describes the contents of the vessels and provides indications and/or instructions regarding administration of contents of the vessels to a subject in need of treatment with said kit(s). The kit form is particularly advantageous when the components are contained in different vessels for administration in different dosage amounts or when titration of the individual components of the kit (e.g., VCD, VCD analog, vehicle) is desired by the prescribing physician.

It should be noted that where various embodiments are described by using a given range, the range is given as such merely for convenience and brevity and should not be construed as an inflexible limitation on the scope of the invention. Accordingly, the description of a range should be considered to have specifically disclosed all the possible sub-ranges as well as individual numerical values within that range.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-F depict the effects of diazepam on electrographic status epilepticus (ESE): evidence for a time dependent benzodiazepine resistance. The data are the mean (solid line) and 95% confidence intervals (shaded) of model predictions for each of the treatments normalized to the power at the time of the injection of the vehicle/drug. (FIGS. 1A-D): Electrographic recordings showing effects of 100 mg/kg diazepam at 15 min versus 30 min compared to controls; (FIGS. 1E-F): Group data showing changes in gamma power as a function of time for 100 mg/kg diazepam at 15 min (FIG. 1E) versus 30 min (FIG. 1F). For FIGS. 1A-D, all panels show electrographic activity over several hours in the upper trace, and temporal expansions of 5 sec at 5 min, 30 min, and 2 hr. (FIG. 1A): Diazepam at 100 mg/kg strongly suppressed ESE. Temporal expansions of lower traces in panel at 5 min, 30 min, and 2 hr illustrate the suppression of electrographic activity after administration of DZP. (FIG. 1B) Lack of effect of vehicle administration at 15 minutes; temporal expansions show normal ESE. (FIG. 1C); lack of effect of DZP at 30 min. (FIG. 1D); effects of vehicle at 30 min. Both FIG. 1C and FIG. 1D illustrate normal ESE as illustrated with multi-hour recordings and temporal expansions at 5 min, 30 min, and 2 hr. (FIGS. 1E, F): Effects of diazepam versus vehicle at 15 and 30 min. Differences between the groups were assessed using the nonparametric Mann-Whitney U-test and the dashed lines represent the time points at which there was a significant difference between the groups (p<0.05).

FIGS. 2A-D show a comparison of VPA versus SPD when administered at 30 min. Lack of effect of vehicle at 30 min. (FIG. 2B); administration of VPA at 30 min even at 300 mg/kg had no detectable effect on ESE, 19 whereas SPD (FIG. 2C) at 130 mg clearly suppressed ESE when administered at 30 min after the first seizure; plot of gamma power as a function of time for SPD versus VPA at 30 min (FIG. 2D).

FIGS. 3A-D show a comparison of effects of SPD at 130 mg/kg and 180 mg/kg at different times after onset of the first seizure. SPD strongly suppressed ESE at 30 min with a dose of 130 mg/kg (FIG. 3A) and still had effects at 45 min at this dose (FIG. 3B). At 60 min, however, 130 mg/kg SPD had no effect (FIG. 3C), whereas ESE was strongly suppressed when the dose was raised to 180 mg/kg (FIG. 3D). Note the increased electrographic activity when ESE was administered at 130 mg/kg as of roughly 6 hr after seizure onset.

FIGS. 4A-D show a comparison of SPD with propofol and pentobarbital. Electrographic data show that SPD (180 mg/kg) (FIG. 4A), propofol (100 mg/kg) (FIG. 5B), and pentobarbital (30 mg/kg) (FIG. 4C) all strongly suppressed ESE when administered 60 min after the first seizure. The plot of power in the gamma band clearly shows that all three compounds were highly effective at 60 min (FIG. 4D).

FIGS. 5A-C demonstrate that VCD suppressed ESE when administered at 30 min (FIG. 5A); electrographic data illustrating the effect of VCD on responders at 30 min after onset of ESE (FIGS. 5B and C); plot of gamma power showing the effect of VCD (180 mg/kg) relative to vehicle (FIG. 5B) and to vehicle and SPD (130 mg/kg), (FIG. 5C) when administered at 30 min.

FIG. 6 depicts a synopsis of the soman-induced seizure (SE) model procedure showing the steps included in the delayed treatment seizure model.

FIGS. 7A-B depict anticonvulsant dose-response curve of valnoctamide (VCD) administered 20 min (FIG. 7A) and 40 min (FIG. 7B) after seizure onset of soman-induced seizures in rats.

FIG. 8 depicts latency for seizure control—the time from when valnoctamide (VCD) was administered to rats until the last epileptiform event could be detected on the EEG record. There is a shorter latency at the 20-min treatment time than the 40-min treatment time.

### DETAILED DESCRIPTION OF THE INVENTION

### Materials and Methods

Materials

Saline (0.9% NaCl) injection, USP, was purchased from Cutter Labs, Inc. (Berkeley, Calif.). The oxime HI-6 DiCl (1-(((4-(aminocarbonyl)pyridinio) methoxy)methyl)-2-((hydroxyimino)methyl)pyridinium dichloride) was obtained from the depository at the Division of Experimental Therapeutics, Walter Reed Army Institute of Research (Silver Spring, Md.). The oxime pyridine-2-aldoxime methylchloride (2-PAM) was purchased from Ayerst Labs, Inc. (New York, N.Y.). Atropine sulfate and atropine methyl nitrate were purchased from Sigma-Aldrich Chemical Company (St. Louis, Mo.). Attane™ (isoflurane, USP) was purchased from Minrad, Inc. (Bethlehem, Pa.). Buprenorphine HCl was purchased from Reckitt Benckiser Pharmaceuticals, Inc. (Richmond, Va.). Diazepam was purchased from T.W. Medical Co. (Lago Vista, Tex.). The nerve agent soman was obtained from the US Army Edgewood Chemical Biological Center (Aberdeen Proving Ground, MD). HI-6 (250 mg/ml), atropine methyl nitrate (4.0 mg/ml), atropine sulfate (0.2 mg/ml) admixed with 2-PAM (50.0 mg/ml) and soman (360 ug/ml) were prepared in saline, either fresh daily (HI-6, atropine methyl nitrate, atropine sulfate+2-PAM) or as frozen aliquots (soman). VCD was received and maintained at room temperature in a dessicator until use. VCD was prepared in multisol (40% propylene glycol, 10% ethanol, 1.5% benzyl alcohol, 48.5% sterile water) to a concentration of 25 mg/mL.

### Animal Care, Surgery and Electrode Implantation

All procedures were performed with protocols approved by the University of Utah Animal Care and Use Committee and in accordance with NIH guidelines for the care and use of laboratory animals. Before and after surgery, the rats (housed individually in temperature (21+20 C) and humidity (50+10%) controlled quarters) were maintained on a 12 h light/dark cycle and fed standard rat chow ad libitum. Male, Sprague-Dawley rats (180-220 g; n=193 or 250-300 g; n=199) were anesthetized with isoflurane (2%) and placed in a stereotaxic unit. The rats were then implanted with bipolar electrodes (M5333-3-B, Plastics One, Roanoke, Va.) for surface cortical recordings. Two holes (500 μm) were drilled on the right side of the midline, and one lead placed into each of the craniotomies to provide differential recordings. A third lead was placed in another craniotomy left of the midline as a ground electrode. The electrodes were fixed in place with dental cement and the skin sutured around the skull. After recovery from the surgery (>7 days), status epilepticus was induced with lithium-pilocarpine treatment.

### Video and EEG Recording

Rats with implanted electrodes were put into custom-built Plexiglas recording chambers equipped with swivel commutators, and were then connected to spring-covered EEG cables (Plastics One, Roanoke, Va.) via their skull caps for EEG recording. Signals were amplified with EEG100C amplifiers (high-pass filter, 1 Hz; low-pass filter, 100 Hz; 5000× gain), digitized at 500 Hz with an MP150 digital-analog converter, and acquired with AcqKnowledge acquisition software (BioPac Systems Inc.; Santa Barbara, Calif.). The tethered rats were also continuously video monitored using an eight-camera infra-red surveillance system linked to a multiplexer so that eight animals could be recorded for 24 h on DVD players (DMR-E520, Panasonic).

### Pilocarpine and Drug Treatment

After recovery from surgery, the implanted rats were pretreated 18-24 h before pilocarpine with intraperitoneal (IP) administration of LiCl (127 mg/kg). Scopolamine bromide (1 mg/kg, IP) was then administered 30 min before injection of pilocarpine (50 mg/kg, IP). At various times after the occurrence of the first motor seizure, vehicle (multisol or methylcellulose), DZP (100 mg/kg), SPD (130 mg/kg or 180 mg/kg), VCD (180 mg/kg), propofol (100 mg/kg) or pentobarbital (30 mg/kg) was injected IP. The test compound was coded so that the experiments were performed with blind procedures. These experiments were performed within the Counter ACT component of the Anticonvulsant Screening Program to identify potential new treatments for pilocarpine-induced ESE.

In some experiments, the animals were anesthetized with isoflorane (5% induction; 3-1.5% maintenance, with oxygen) and placed in a stereotaxic instrument. Two stainless steel screws were placed in the skull bilaterally midway between bregma and lamda and ˜3 mm lateral to the midline. A third screw was placed over the cerebellum. The screws were connected to a miniature connector with wires and the screws, wires and connector were then anchored to the skull with dental cement. The incision was sutured; the animal was removed from the frame, given the analgesic buprenorphine HCl (0.03 mg/kg, SC) and placed on a warming pad for at least 30 min before being returned to the animal quarters. Approximately seven days elapsed between surgery and experimentation.

### Anticonvulsant Test

Animals were typically tested in squads of eight on a given study day. The animals were randomized among treatment groups each test day. The animals are weighed, placed in individual recording chambers and connected to the recording apparatus. EEG signals were recorded using CDE 1902 amplifiers and displayed on a computer running Spike2 software (Cambridge Electronic Design, Ltd., Cambridge, UK). Baseline EEG was recorded for at least 20 min. The animals were then pretreated with 125 mg/kg, IP, of the oxime HI-6 to prevent the rapid lethal effects of the soman challenge. Thirty min later the animals were challenged with 180 ug/kg, SC, soman (1.6×LD50) and 1 min later treated with 2.0 mg/kg, IM, atropine methyl nitrate to inhibit peripheral secretions. The animals were then closely monitored both visually and on the EEG for seizure onset. Seizure onset was operationally defined as the appearance of >10 sec of rhythmic high amplitude spikes or sharp waves that were at least twice the baseline amplitude accompanied by a rhythmic bilateral flicking of the ears, facial clonus and possibly forepaw clonus. At 5 or 20 min after seizure onset, the animals received standard medical countermeasures: 0.1 mg/kg atropine sulfate+25 mg/kg 2-PAM Cl admixed to deliver 0.5 ml/kg, IM, and 0.4 mg/kg IM diazepam. These standard medical countermeasures are insufficient, by themselves, to terminate soman-induced seizures.

Immediately after administering the standard medical countermeasures, individual animals received an IP dose of VDC. The animals were monitored for at least 5 hr after exposure and then returned to the animal housing room. Twenty-four hr after the exposure, the surviving animals were weighed and the EEG again recorded for at least 30 min. Following this, the animals were administered an anesthetic dose (75 mg/kg, IP) of pentobarbital and when deeply anesthetized were perfused intracardially with saline followed by formalin. Evaluation and categorization of the EEG response by an individual animal to treatment were performed by a technician and investigator, both well-experienced with the appearance of nerve agent-induced EEG seizure activity. The overall rating and timing of different events required consensus between both individuals, who were aware of the treatment conditions of an individual animal. To be rated as having the seizure terminated, all spiking and/or rhythmic waves had to stop and the EEG had to remain normal at all subsequent observation times (n.b., throughout the 5-hr record following exposure and for the 30-min record 24 hr later). For each animal in which the seizure was terminated, the latency to seizure termination was measured as the time from when the animal received the VCD treatment to the last observable epileptiform event in the EEG.

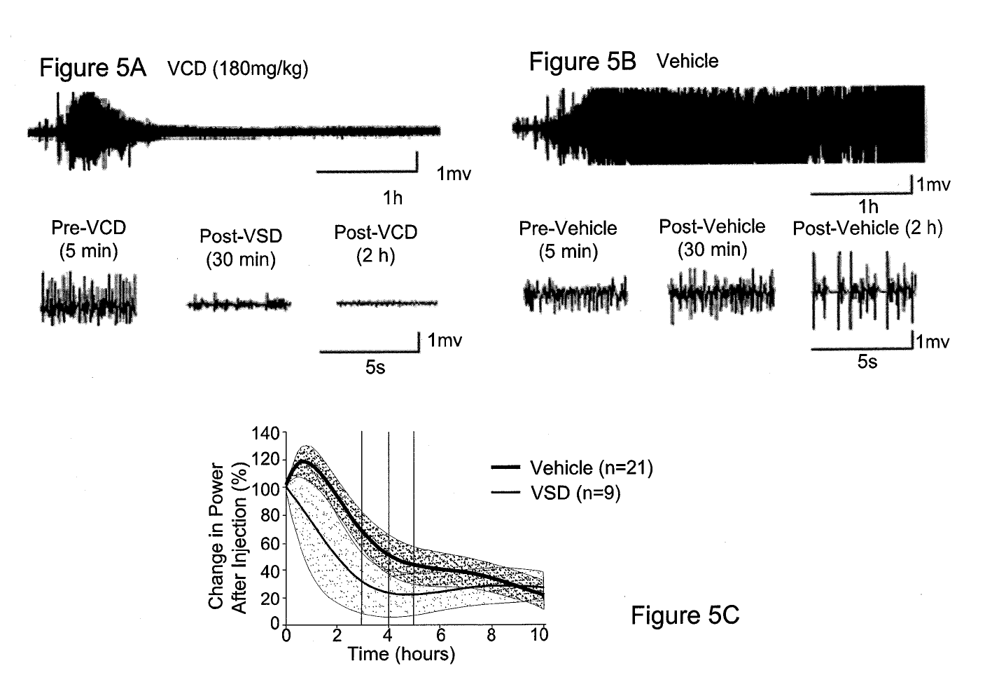

An exemplary experimental anticonvulsant test procedure (soman-induced seizure SE models) is outlined in FIG. 6. In this delayed treatment model, Sprague-Dawley rats surgically prepared for EEG recording were pretreated with 125 mg/kg, I.P, HI-6 (4-aminocarbonyl)pyridinio]methoxy]methyl]-2[(hydroxyimino)methyl]pyridinium dichloride) and then challenged 30 min later 180 ug/kg, S.C, soman and given 2.0 mg/kg, I.M, methyl atropine. Treatment was initiated 5, 20 or 40 minutes after seizure onset: atropine sulphate (0.45 mg/kg)+2PAM (25 mg/kg)+diazepam (2.0 mg/kg)+test anticonvulsant (4-5 doses) Ns=4-6/dose). EEG was monitored for ˜6 hr and for 30 minutes on the next day.

### Data Analysis

The EEG data from 0-10 h after the administration of the test drug was band-pass filtered (20-70 Hz) and the power spectral density calculated and plotted over time. To compare across groups, the energy data were fit with an 8th-order polynomial, and statistical analyses were performed at different times after onset of SE [3]. Differences between the groups were assessed using the non-parametric Mann-Whitney U-test or Kruskal Wallis followed by a Dunn's multiple comparison test.

### Results

Time-dependent Effects of Diazepam (DZP) and Valproic Acid (VPA) on Electrographic Status Epilepticus (ESE)

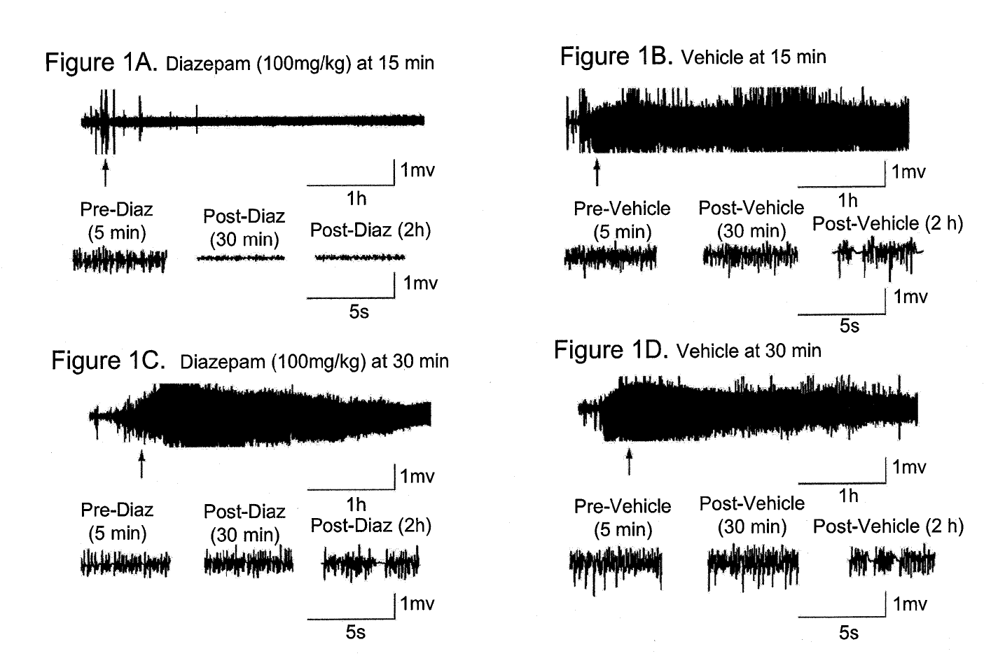

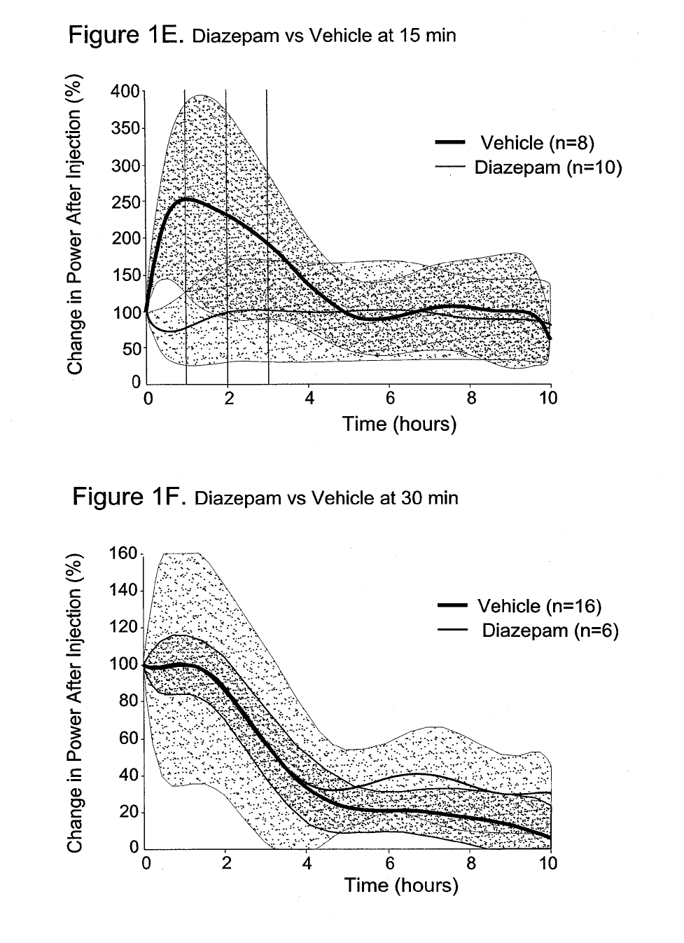

Although benzodiazepines such as DZP demonstrate efficacy when administered soon after the onset of SE, this class of compounds generally fails to stop seizure activity when administered more than 30 min after seizure onset). Thus, the initial experiments aimed to establish that DZP shows efficacy when administered at 15 min in this model under the present experimental conditions, but lacks efficacy for suppression of ESE at 30 min. FIG. 1 shows that when DZP was injected at 15 min, the electrographic activity was suppressed for several hours (FIG. 1A). The efficacy of DZP at 15 min was apparent at lower doses (i.e., 10-100 mg/kg), but at 30 min after the occurrence of the first motor seizure, DZP had virtually no effect on ESE, even at 100 mg/kg. Similarly, VPA at 300 mg/kg also had no detectable effect on ESE (FIG. 2B). Thus, two standard-of-care AEDs, even at high doses, had little or no detectable effect on ESE.

### The Effects of SPD on ESE: Dosage and Time-Course

SPD has a broad spectrum of anticonvulsant activity against several electrically- and chemically-induced seizure models in mice and rats with potent ED50 values (18-29 mg/kg) and with wide protective indexes (PI=TD50/ED50) of 4.4-7.7 [7]. In behavioral studies, SPD had potent anticonvulsant activity in the rat model of pilocarpine-induced status epilepticus. When SPD was administered 30 min after the first pilocarpine-induced seizure, it had ED50 and ED97 values of 84 mg/kg and 149 mg/kg, respectively. SPD (100-174 mg/kg) also protected against seizures for 4-8 h after exposure to the nerve agent soman when administered to rats and guinea pigs 20 min or 40 min after onset of SE [7]. Since SPD is a chiral compound with two asymmetric centers, the racemic-SPD tested so far is a mixture of four individual stereoisomers.

Thus, it was previously found that that administration of SPD at 0 and 30 min in the lithium-pilocarpine model suppressed convulsive seizures. Other experiments in this previous study provided electrographic evidence for efficacy in two different animal models of nerve-agent exposure. In these previous experiments, ED50 ranged from 65 mg/kg to 149 mg/kg in the various animal models for different times of administration and outcome measures.

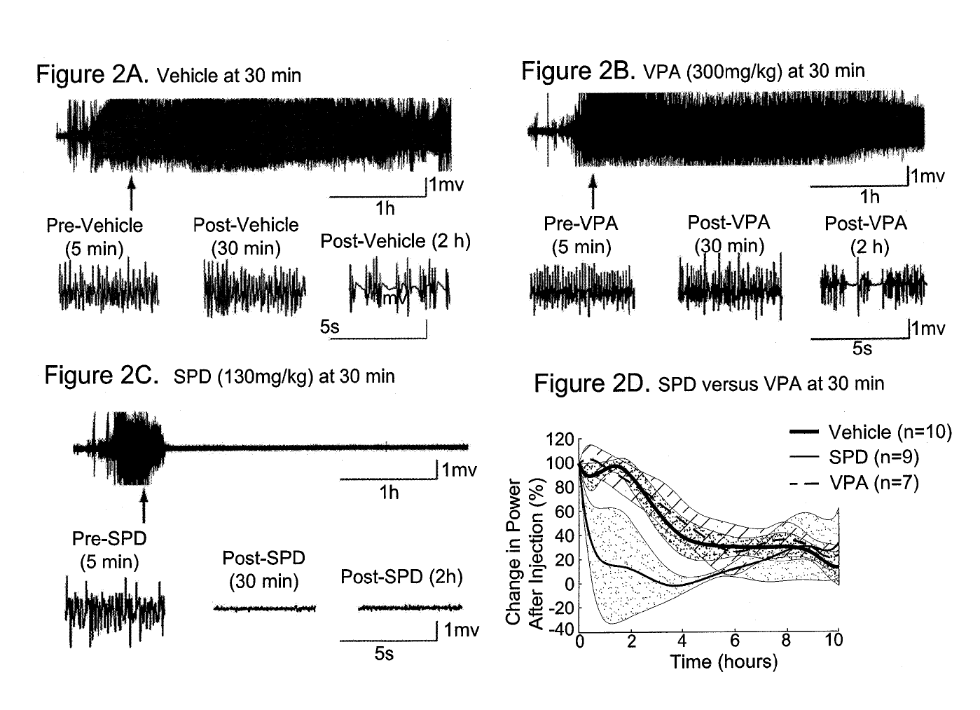

The raw electrographic data (FIG. 2) and the quantitative analysis of group data (FIG. 3A) both show a clear effect of 130 mg/kg SPD on EEG power in the γ-band, when SPD was administered at 30 min. A diminished effect was observed when this dose of SPD was administered at 45 min (FIG. 3B), and no effect was detected with SPD administration at 60 min after the first motor seizure (FIG. 3C). However, a powerful effect on ESE was found when SPD was administered at 60 min if the dose of SPD was increased about 50% from 130 mg/kg to 180 mg/kg (FIG. 3D). The effects of SPD persisted for several hours, but under some conditions could show a rebound effect between 7-10 hr (FIG. 3A-C). Thus, the effect of SPD progressively decreased as the time of administration was increased from 30 min to 45 min to 60 min, but a 50% increase in dose led to a profound effect of SPD, when administered at 60 min.

Comparison of Effects of SPD with the Anesthetics Propofol and Pentobarbital

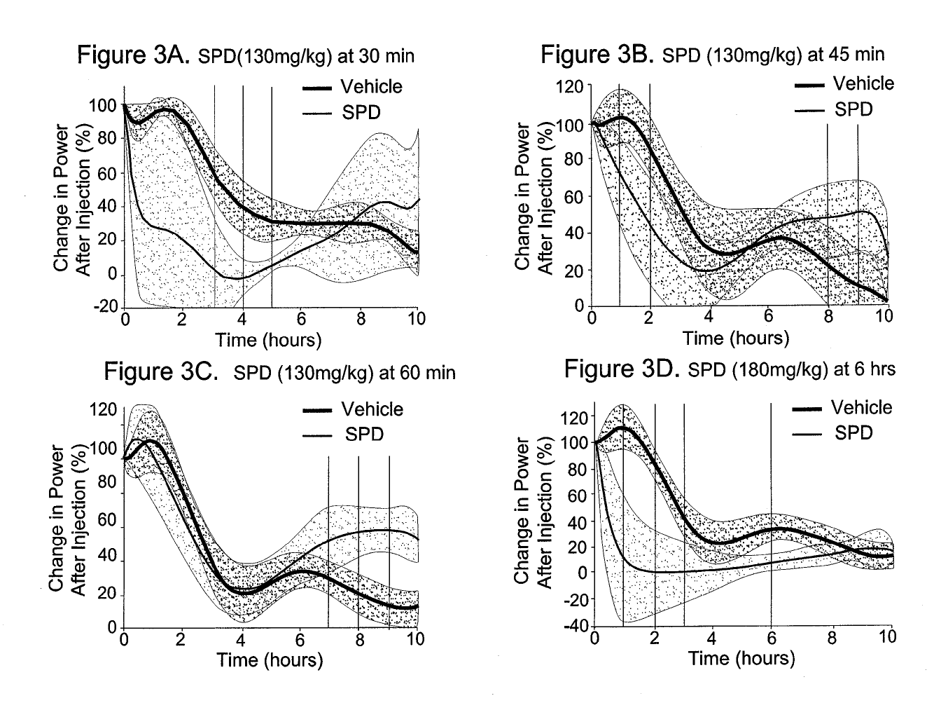

When ESE cannot be suppressed by first or second-line AEDs, anesthetics such as propofol and pentobarbital are frequently used as third-line therapy to block the electrographic seizures of refractory ESE. Accordingly, SPD, propofol and pentobarbital were compared in regard to their efficacy to suppress ESE (FIG. 4). All three compounds greatly reduced the mean power of the EEG when administered 60 min after the first motor seizure. Therefore, SPD appeared to have suppressive effects comparable to propofol and pentobarbital, in terms of its ability to suppress severe pilocarpine-induced ESE (FIG. 4), which was previously shown to be refractory to 100 mg/kg DZP by 30 min after the first motor seizure (FIG. 1).

### The Effect of Valnoctamide (VCD) on ESE

VCD is a constitutional isomer of VPA that corresponds to the amide, valpromide (VPDB), an eight-carbon homologue (i.e., one-less carbon) of SPD. In previous studies using behavioral measures of convulsive seizures during pilocarpine-induced SE, VCD showed efficacy at 0 min (65 mg/kg), but not at 30 min (80 mg/kg). This study described efficacy of VCD in acute seizure models based on maximum electroshock and metrazol, and the ED50 of VCD appeared to be qualitatively similar to SPD [7].

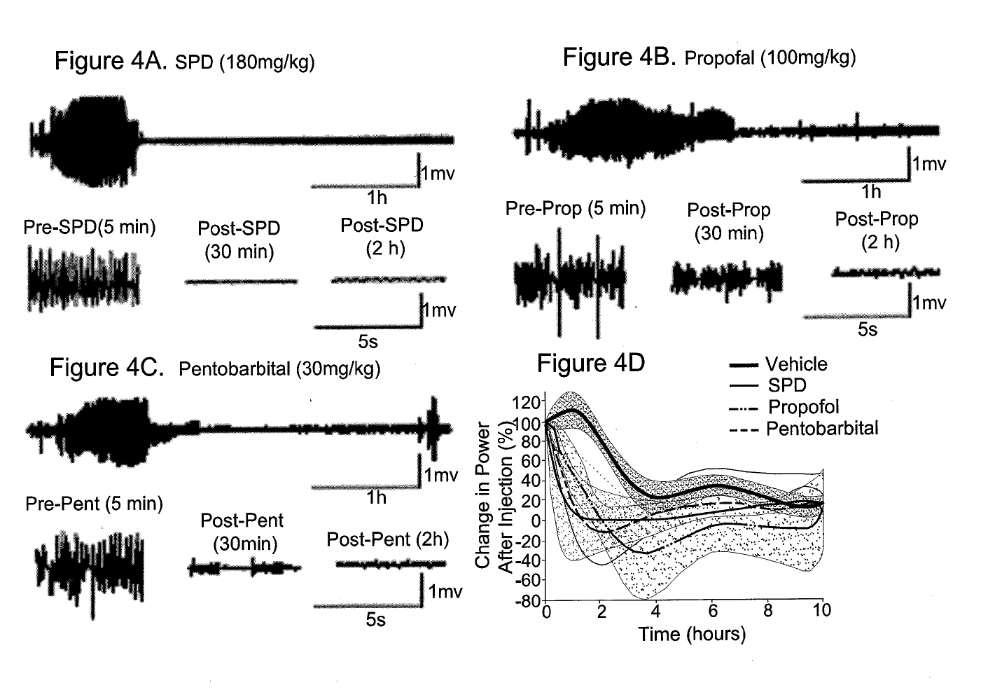

The effect of VCD on ESE was tested at a relatively high dose (180 mg/kg) at 30 min after the first motor seizure, and VCD clearly suppressed EEG power in the γ-band during ESE (FIG. 5).

Thus, when administered at 30 min—a time when DZP had no detectable effect on ESE at a dose of 100 mg/kg—VCD suppressed ESE, when the dose was raised to 180 mg/kg (FIGS. 5A and C). Because no obvious deleterious effects of VCD were observed (e.g., no evidence of increased mortality), these data show that VCD demonstrates efficacy against ESE.

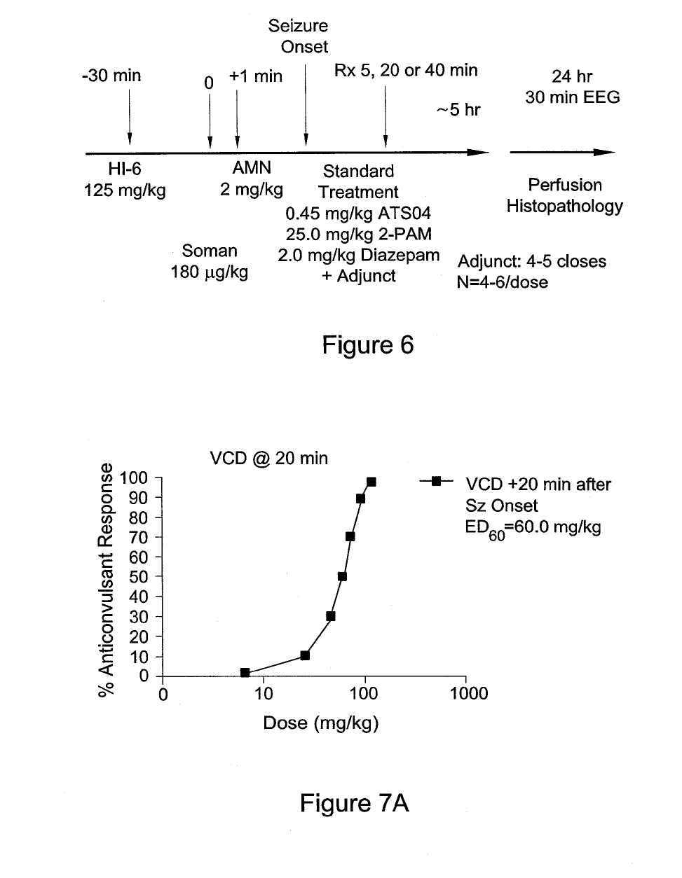

FIG. 7 shows anticonvulsant dose-response curve of VCD administered 20 and 40 min after SE seizure onset and shows that the ED50 values are almost identical at treatment delay times of 20 and 40 min (ED50=60 mg/kg at 20 min and 62 mg/kg at 40 min delay time).

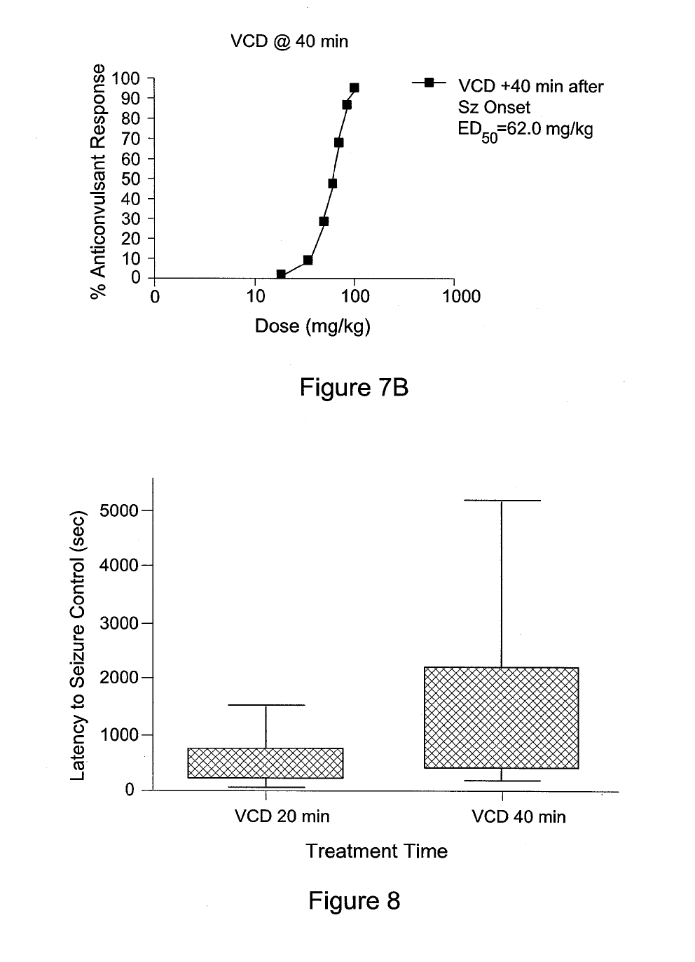

In FIG. 8 the latency for seizure control, i.e., the time from when VCD was administered to rats until the last epileptiform event could be detected on the EEG record is shown. Evidently, there is a shorter latency at the 20 min treatment time than the 40 min treatment time. A rapid seizure control was observed at 20 min treatment delay being shorter that the time for seizure control at 20 min.

Table 1 shows a test of the anticonvulsant activity of SPD and VCD compounds in the rat nerve agent seizure model for correlating anticonvulsant efficacy with potential neuroprotectant effect. The ED₅₀ values for anticonvulsant effect and latencies for seizure control at different treatment delay times are shown.

TABLE 1
ED50 values for anticonvulsant effect and latencies for seizure
control at different treatment delay times (5 min; 20 min and 40 min).

| Compound | 5 min delay | 20 min delay | 40 min delay |
|---|---|---|---|
| (VCD) | 25.8 mg/kg | 60.0 mg/kg | 62.0 mg/kg |
| (SPD; 2R, 3R) | not provided | 40.5 mg/kg | |
| (SPD; 2R, 3S) | not provided | 69.0 mg/kg | not provided |

From Table 1 it can be seen that only VCD had a robust anticonvulsant effects at all three test delay times. Thus, it was observed that the anticonvulsant ED50 for VCD was 25.8 at 5 min treatment delay, 60.0 mg/kg at 20 min treatment delay and 62.0 mg/kg at 40 min treatment delays.

The herein described experiments established that DZP, even at 100 mg/kg, had virtually no effect on ESE. Similarly, VPA at 300 mg/kg also had no detectable effect. Thus, the present studies were demonstrably performed under conditions where ESE could be considered refractory to first- and second-line, standard of-care therapies. In addition, increasing the dose of SPD from 130 to 180 mg/kg led to powerful suppressive effects on ESE, even when administered at 60 min, which were not apparent at the lower dose. The suppressive effect of SPD on ESE lasted for several hours, but under some conditions showed a rebound effect between 7-10 hr. VCD, a homolog of SPD, also clearly suppressed ESE when administered at 30 min. These data indicate that SPD has potential at high doses to strongly suppress benzodiazepine-resistant ESE, even when administered as late as 60 min after the first seizure.

At sufficiently high doses, the effects of SPD on ESE lasted for 6-8 h, so the effects of SPD not only have a rapid onset, but in theory they persist long enough to allow time for subsequent treatment with other countermeasures against SE induced by nerve agents. An analysis of the dosage is also obviously critical, particularly since doses that might be appropriate for chronic treatment of epilepsy (i.e., with minimal side effects) may be too low for appropriate treatment of status epilepticus, where deficits in motor and cognitive performance are relatively unimportant compared to systemic physiological effects, such those that may involve the respiratory or cardiovascular systems.

### Benzodiazepine-Resistance of Pilocarpine-Induced Status Epilepticus

A critical first step in this analysis was to establish that the effects of a prototypical benzodiazepine lacked efficacy for suppression of ESE at 30 min. The electrographic data showed that DZP at 10-100 mg/kg had no consistent detectable effect on ESE. As a positive control, DZP showed efficacy at 15 min, which is consistent with previous studies demonstrating that the effects of DZP are both dose- and time-dependent). The data, which show a time-dependence of the effect of DZP on ESE and a lack of effect of DZP when administered at 30 min, set the stage for subsequent studies in which efficacy at 30 min and longer times after onset of pilocarpine-induced seizures is considered to represent benzodiazepine-resistant ESE.

### Actions of SPD: Dose and Time Effects

A recent study showed that administration of SPD in lithium pilocarpine-treated rats strongly suppressed behavioral seizures when administered at 0 and 30 min, and other experiments showed electrographic data for efficacy in two nerve agent models [7]. The ED50 values for SPD in these studies ranged between 65 mg/kg and 149 mg/kg for different models, times of administration, and outcome measures. The data presented here showed a powerful effect of 130 mg/kg SPD at 30 min, with a diminished effect at 45 min, and no effect at 60 min after the first motor seizure. When 130 mg/kg SPD was administered at 45 min, the effect persisted for only 3-4 h, compared to 6-8 hr when SPD was administered at 30 min. However, under both conditions, a rebound effect occurred between 6-10 h after SPD administration. When the dose was increased by about 50% to 180 mg/kg, SPD had a dramatic effect at 60 min, and the effects persisted for 7-8 hr.

### Effects of VPA and VCD on ESE

VPA is considered a second-line therapy for benzodiazepine-refractory SE, and furthermore, the widespread usefulness of VPA as an AED for several seizure types has led to the development of several VPA analogs, of which VCD has probably been the most widely studied. As part of the initial experiments to assess the level of resistance of ESE to standard AEDs, we found that 300 mg/kg VPA had no effect on ESE when administered at 30 min. In previous studies using behavioral measures of convulsive seizures in the lithium-pilocarpine model [7], relatively low doses of VCD showed efficacy at 0 min (65 mg/kg), but not at 30 min (80 mg/kg).

1. A method of treating benzodiazepine-resistant status epilepticus (SE) in a subject having been exposed to a nerve agent inducing said SE, the method comprising administering to the subject a therapeutically effective amount of valnoctamide (VCD) or a pharmaceutically acceptable salt thereof, wherein said VCD is administered after the subject has experienced at least one SE episode indicative of exposure to said agent.

2. The method according to claim 1, wherein the VCD is administered 30 minutes or more after said subject has experienced at least one SE episode indicative of exposure to said agent.

3. The method according to claim 1, wherein the therapeutically effective amount of VCD is more than the human equivalent of 80 mg/kg in rats.

4. A method of treating benzodiazepine resistant status epilepticus (SE) in a subject having been exposed to a nerve agent inducing said SE, the method comprising administering to the subject a therapeutically effective amount of valnoctamide (VCD) or a pharmaceutically acceptable salt thereof, wherein the therapeutically effective amount of VCD is more than the human equivalent of 80 mg/kg in rats.

5. The method according to claim 4, wherein the VCD is administered 30 minutes or more after said subject has experienced at least one SE episode indicative of exposure to said agent.

6. A method of treating benzodiazepine-resistant status epilepticus (SE) in a subject having been exposed to a nerve agent inducing said SE, the method comprising administering to the subject a therapeutically effective amount of valnoctamide (VCD) or a pharmaceutically acceptable salt thereof, wherein said VCD is administered after said subject has experienced at least one SE episode indicative of exposure to said agent, and wherein the therapeutically effective amount of VCD is more than the human equivalent of 80 mg/kg in rats.

7. The method according to claim 4, wherein the VCD is administered 30 minutes or more after said subject has experienced at least one SE episode indicative of exposure to said agent.

8. The method of any one of claims 1 to 7, wherein SE is induced by at least one nerve agent selected from the group consisting of tabun (GA), sarin (GB), soman (GD), cyclosarin (GF), 2-(dimethylamino)ethyl N,N-dimethylphosphor amidofluoridate (GV), a Novichok agent, S-(diethylamino)ethyl O-ethylethyl phosphonothioate (VE), O,O-diethyl 5-[2-(diethylamino)ethyl]phosphorothioate (VG), 2-(ethoxymethyl phosphoryl) sulfanyl-N,N-diethylethanamine (VM) and ethyl({2-[bis(propan-2-yl)amino]ethyl}sulfanyl)(methyl)phosphinate (VX).

9. The method of any one of claims 1 to 8, wherein SE is not pilocarpine or lithium-pilocarpine induced SE.

10. The method of any one of claims 1 to 9, wherein the therapeutically effective amount of VCD is between the human equivalent of about 100 mg/kg and about 500 mg/kg in rats.

11. The method of claim 10, wherein the therapeutically effective amount of VCD is between the human equivalent of about 100 mg/kg and about 200 mg/kg in rats.

12. The method of claim 11, wherein the therapeutically effective amount of VCD is the human equivalent of about 180 mg/kg in rats.

13. A pharmaceutical composition comprising as an active agent a therapeutically effective amount of valnoctamide (VCD) or a pharmaceutically acceptable salt thereof for use in treating benzodiazepine resistant status epilepticus (SE) in a subject having been exposed to a nerve agent inducing said SE, wherein said VCD is administered after said subject has experienced at least one SE episode indicative of exposure to said agent.

14. The composition according to claim 13, wherein the VCD is administered 30 minutes or more after said subject has experienced at least one SE episode indicative of exposure to said agent.

15. A pharmaceutical composition, comprising as an active agent a therapeutically effective amount of valnoctamide (VCD) or a pharmaceutically acceptable salt thereof for use in treating benzodiazepine resistant status epilepticus (SE) in a subject having been exposed to a nerve agent inducing said SE, wherein the therapeutically effective amount of VCD is more than the human equivalent of 80 mg/kg in rats.

16. A pharmaceutical composition comprising as an active agent a therapeutically effective amount of valnoctamide (VCD) or a pharmaceutically acceptable salt thereof for use in treating benzodiazepine resistant status epilepticus (SE) in a subject having been exposed to a nerve agent inducing said SE, wherein said VCD is administered after said subject has experienced at least one SE episode indicative of exposure to said agent, and wherein the therapeutically effective amount of VCD is more than the human equivalent of 80 mg/kg in rats.

17. The composition according to claim 16, wherein the VCD is administered 30 minutes or more after onset of SE.

18. The composition according to any one of claims 13 to 17, comprising a vehicle in which a therapeutically effective amount of VCD or a pharmaceutically acceptable salt thereof is solubilized, said vehicle comprising between about 0.1 and 0.6 gram/1,000 ml of calcium chloride, between about 0.1 and 0.6 gram/1,000 ml of potassium chloride and between about 2 and 12 gram/1,000 ml of sodium chloride.

19. Use of valnoctamide (VCD) or a pharmaceutically acceptable salt thereof in the preparation of a pharmaceutical composition for treating benzodiazepine resistant status epilepticus (SE) in a subject having been exposed to a nerve agent inducing said SE, wherein said VCD is administered after said subject has experienced at least one SE episode indicative of exposure to said agent.

20. The use according to claim 19, wherein the VCD is administered 30 minutes or more after said subject has experienced at least one SE episode indicative of exposure to said agent.

21. Use of valnoctamide (VCD) or a pharmaceutically acceptable salt thereof in the preparation of a pharmaceutical composition for treating benzodiazepine resistant status epilepticus (SE) in a subject having been exposed to a nerve agent inducing said SE, wherein the therapeutically effective amount of VCD is more than the human equivalent of 80 mg/kg in rats.

22. Use of valnoctamide (VCD) or a pharmaceutically acceptable salt thereof in the preparation of a pharmaceutical composition for treating benzodiazepine resistant status epilepticus (SE) in a subject having been exposed to a nerve agent inducing said SE, wherein said VCD is administered after said subject has experienced at least one SE episode indicative of exposure to said agent, and wherein the therapeutically effective amount of VCD is more than the human equivalent of 80 mg/kg in rats.

23. The use according to claim 22, wherein the VCD is administered 30 minutes or more after said subject has experienced at least one SE episode indicative of exposure to said agent.

24. The use according to any one of claims 19 to 23, wherein said composition comprises a vehicle in which a therapeutically effective amount of VCD or a pharmaceutically acceptable salt thereof is solubilized, said vehicle comprising between about 0.1 and 0.6 gram/1,000 ml of calcium chloride, between about 0.1 and 0.6 gram/1000 of potassium chloride and between about 2 and 12 gram/1,000 ml of sodium chloride.

25. A pharmaceutical composition, suitable for injection, comprising a vehicle in which a therapeutically effective amount of VCD or at least one analog thereof is solubilized, said vehicle comprising between about 0.1 and 0.6 gram/1,000 ml of calcium chloride, between about 0.1 and 0.6 gram/1,000 ml of potassium chloride and between about 2 and 12 gram/1,000 ml of sodium chloride.

26. The composition of claim 25, for use in treating benzodiazepine resistant status epilepticus (SE) in a subject having been exposed to a nerve agent inducing said SE, wherein said VCD or at least one analog thereof is administered 30 minutes or more after said subject has experienced at least one SE episode indicative of exposure to said agent.

27. The composition of claim 25, for use in treating benzodiazepine resistant status epilepticus (SE) in a subject having been exposed to a nerve agent inducing said SE, wherein the therapeutically effective amount of VCD or at least one analog thereof is more than the human equivalent of 80 mg/kg in rats.

28. The composition of claim 25 for use in treating benzodiazepine resistant status epilepticus (SE) in a subject having been exposed to a nerve agent inducing said SE, wherein the therapeutically effective amount of VCD or at least one analog is more than the human equivalent of 80 mg/kg in rats, wherein said VCD is administered 30 minutes or more after said subject has experienced at least one SE episode indicative of exposure to said agent.

29. The composition of any one of claims 25 to 28, wherein SE is induced by at least one agent selected from the group consisting of Tabun (GA), Sarin (GB), Soman (GD), cyclosarin (GF), 2-(Dimethylamino)ethyl N,N-dimethylphosphoramidofluoridate (GV), a Novichok agent, S-(Diethylamino)ethyl O-ethyl ethylphosphonothioate (VE), O,O-diethyl 5-[2-(diethylamino)ethyl]phosphorothioate (VG), 2-(ethoxy-methylphosphoryl) sulfanyl-N,N-diethylethanamine (VM) and ethyl({2-[bis(propan-2-yl)amino]ethyl}sulfanyl)(methyl)phosphinate (VX).

30. The composition of any one of claims 25 to 28, wherein SE is not pilocarpine or lithium-pilocarpine induced SE.

31. The composition of any one of claims 25 to 28, wherein the therapeutically effective amount of VCD or at least one analog thereof is between the human equivalent of about 100 mg/kg and about 500 mg/kg in rats.

32. The composition of claim 31, wherein the therapeutically effective amount of VCD or at least one analog thereof is between the human equivalent of about 100 mg/kg and about 200 mg/kg in rats.

33. The composition of claim 32, wherein the therapeutically effective amount of VCD or at least one analog thereof is the human equivalent of about 180 mg/kg in rats.

34. The composition of any one of claims 25 to 33, said composition being suitable for intramuscular injection, wherein the concentration of VCD or at least one analog thereof in the vehicle is between about 0.5 and 25% by weight in solution.

35. The composition according to any one of claims 25 to 34, wherein said VCD or at least one analog thereof is in lyophilized form for the reconstitution in a sterile solution.
